(19) United States
(12) Patent Application Publication
(10) Pub. No.: US 2023/0176237 A1
(43) Pub. Date: Jun. 8, 2023

(54) X-RAY IMAGING DEVICE

(71) Applicant: Commissariat à l'Énergie Atomique et aux Énergies Alternatives, Paris (FR)

(72) Inventors: François Templier, Grenoble Cedex 9 (FR); Loick Verger, Grenoble Cedex 9 (FR); Eric Gros-Daillon, Grenoble Cedex 9 (FR); Sébastien Becker, Grenoble Cedex 9 (FR)

(73) Assignee: Commissariat à l'Énergie Atomique et aux Énergies Alternatives, Paris (FR)

(21) Appl. No.: 17/992,988

(22) Filed: Nov. 23, 2022

(30) Foreign Application Priority Data
Dec. 2, 2021 (FR) 2112879

(51) Int. Cl. G01T 1/24; G01T 1/20

(52) U.S. Cl. CPC G01T 1/241; G01T 1/20181

(57) ABSTRACT

An X-ray imaging device, including: a transfer substrate including electric connection elements; an array of pixels, each including a monolithic elementary chip bonded and electrically connected to elements of electric connection of the transfer substrate, and a direct conversion X photon detector electrically connected to the elementary chip, wherein, in each pixel, the elementary chip includes an integrated circuit for reading from the detector of the pixel.

### CROSS REFERENCE TO RELATED APPLICATIONS

This application claims priority to French application number 2112879, filed Dec. 2, 2022, the contents of which is incorporated herein by reference in its entirety.

### TECHNICAL BACKGROUND

The present disclosure concerns an X-ray imaging device and a method of manufacturing such a device, particularly for radiography applications, for example, in the field of medical imaging.

### PRIOR ART

Among known X-ray imaging devices, indirect conversion devices and direct conversion devices can be distinguished.

Indirect conversion devices comprise an array of photodiodes adapted to capturing a light radiation, and a scintillator arranged above the array of photodiodes. In operation, the scintillator emits light as a result of the absorption of the X-rays. The light emitted by the scintillator is converted into electric charges by the photodiodes. Thus, the array of photodiodes acquires an image representative of the light distribution emitted by the scintillator, this light distribution being itself representative of the X-ray distribution received by the scintillator.

Direct conversion devices comprise a layer of a semiconductor conversion material adapted to directly converting the absorbed X-rays, into electric charges. The conversion layer is arranged above an array of elementary circuits adapted to reading the electric charges generated in the conversion material. In operation, the conversion layer generates electric charges as a result of the absorption of the X-rays. These charges are read by the array of readout circuits. Thus, the array of readout circuits directly acquires an image representative of the X-ray distribution received by the conversion material.

The forming of direct conversion X-ray imaging devices is here more particularly considered.

### SUMMARY OF THE INVENTION

An embodiment provides an X-ray imaging device comprising:

a transfer substrate comprising electric connection elements;

an array of pixels, each comprising a monolithic elementary chip bonded and electrically connected to elements of electric connection of the transfer substrate, and a direct conversion X photon detector electrically connected to the elementary chip,

wherein, in each pixel, the elementary chip comprises an integrated circuit for reading from the detector of the pixel.

According to an embodiment, in each elementary chip, the integrated circuit for reading from the detector of the pixel is formed in CMOS technology.

According to an embodiment, in each pixel, the detector comprises an active detection stack based on a semiconductor material adapted to directly converting X photons into electric charges, for example, a material from the group comprising amorphous selenium (a:Se), gallium arsenide (GaAs), mercury iodide (HgI₂), lead oxide (PbO), cadmium telluride (Cd(Zn)Te), or a perovskite material.

According to an embodiment, the active detection stack continuously extends over the entire surface of the pixel array.

According to an embodiment, in each pixel, the detector comprises a lower electrode and an upper electrode, the upper electrode being common to all the pixels of the device and the lower electrode being individualized per pixel.

According to an embodiment, in each pixel, the detector covers the elementary chip of the pixel.

According to an embodiment, in each pixel, the elementary chip of the pixel comprises an inorganic LED and an integrated circuit for controlling the LED.

Another embodiment provides an assembly comprising first and second stacked X-ray imaging devices such as defined hereabove.

According to an embodiment, the assembly comprises a filtering layer between the first and second devices.

Another embodiment provides a method of manufacturing an X-ray imaging device such as defined hereabove, wherein the elementary chips are collectively transferred and bonded to the transfer substrate, by means of a temporary support substrate.

According to an embodiment, the method comprises a step of deposition of a planarization layer after the transfer and the bonding of the elementary chips onto the transfer substrate.

According to an embodiment, the method comprises a step of transfer of the detectors onto the upper surface of the planarization layer.

### BRIEF DESCRIPTION OF THE DRAWINGS

The foregoing and other features and advantages will be discussed in detail in the following non-limiting description of specific embodiments in connection with the accompanying drawings, in which:

FIGS. 1A, 1B, 1C, 1D, 1E, and 1F are top and cross-section views illustrating steps of an example of a method of manufacturing an X-ray imaging device according to an embodiment;

FIG. 2 is a cross-section view illustrating an alternative embodiment of the method of FIGS. 1A, 1B, 1C, 1D, 1E, and 1F;

FIGS. 3A, 3B, 3C, 3D, and 3E are cross-section views illustrating steps of an example of a method of manufacturing elementary pixel chips of an X-ray imaging device according to an embodiment;

FIGS. 4A and 4B are top and cross-section views illustrating steps of another example of a method of manufacturing an X-ray imaging device according to an embodiment; and

FIG. 5 is a cross-section view illustrating a variant of an X-ray imaging device according to an embodiment.

### DESCRIPTION OF THE EMBODIMENTS

Like features have been designated by like references in the various figures. In particular, the structural and/or functional features that are common among the various embodiments may have the same references and may dispose identical structural, dimensional and material properties.

For the sake of clarity, only the steps and elements that are useful for an understanding of the embodiments described herein have been illustrated and described in detail. In particular, the various possible applications of the described X-ray imaging devices have not been detailed, the described embodiments being compatible with all or most known X-ray imaging applications, and more particularly applications capable of taking advantage of X-ray imaging devices of large dimensions, for example, devices having lateral dimensions greater than 10 cm and preferably greater than 20 cm. Further, the forming of the direct conversion detectors and of the electronic control circuits of the described devices have not been shown, the forming of these elements being within the abilities of those skilled in the art based on the indications of the present disclosure. By X-rays, there is here meant, for example, radiations formed of photons having an energy in the range, for example, from 1,000 eV (electron-volts) to 20 MeV (mega-electron-volts).

Unless indicated otherwise, when reference is made to two elements connected together, this signifies a direct connection without any intermediate elements other than conductors, and when reference is made to two elements coupled together, this signifies that these two elements can be connected or they can be coupled via one or more other elements.

In the following description, when reference is made to terms qualifying absolute positions, such as terms “front”, “back”, “top”, “bottom”, “left”, “right”, etc., or relative positions, such as terms “above”, “under”, “upper”, “lower”, etc., or to terms qualifying directions, such as terms “horizontal”, “vertical”, etc., it is referred unless specified otherwise to the orientation of the cross-section views of the drawings.

Unless specified otherwise, the expressions “around”, “approximately”, “substantially” and “in the order of” signify within 10%, and preferably within 5%.

According to an aspect of the described embodiments, there is provided an X-ray imaging device comprising a transfer substrate and an X-ray detection pixel array formed on the transfer substrate. Each detection pixel comprises a detector based on a semiconductor conversion material adapted to directly converting X photons into electric charges, formed on the transfer substrate and electrically coupled or connected to electric connection elements (track, area, terminals or pads of electric connection) of the transfer substrate, and a monolithic elementary chip, bonded and electrically connected to elements of electric connection of the transfer substrate. In each pixel, the elementary chip is connected to the X-ray detector, for example by at least one element of electric connection of the transfer substrate. The elementary chip comprises at least one integrated circuit for reading from the X-ray detector, preferably formed in CMOS technology.

Each elementary chip comprises a connection surface comprising a plurality of electric connection pads (also called terminals or landings) intended to be connected to the transfer substrate for the chip control. Each elementary chip comprises a connection surface comprising a plurality of electric connection pads (also called terminals or landings) intended to be connected to the transfer substrate for the chip control. The chips are transferred onto the transfer substrate, with their connection surfaces facing the connection surface of the transfer substrate, and bonded to the transfer substrate so as to connect the electric connection pads of each chip to the corresponding electric connection pads of the transfer substrate.

An advantage of the described embodiments is that they enable to obtain imaging devices of large dimensions, for example having lateral dimensions greater than 10 cm, preferably greater than 20 cm, at relatively low costs, while benefiting from the advantages of monolithic integrated circuits, for example, CMOS circuits, for the reading from the X-ray detectors. An advantage particularly lies in the low readout noise introduced by such monolithic integrated circuits with respect to circuits based on TFT (“Thin Film Transistor”) transistors, formed by successive depositions of a plurality of thin layers directly on the transfer substrate. Another advantage is the gain in terms of reading rapidity, due to the better mobility of the charge carriers in such monolithic integrated circuits with respect to TFT circuits. Further, such circuits optionally enable to implement additional functions of processing of the electric signals delivered by the X-ray detectors. Another advantage lies in the low bulk of monolithic elementary chips with respect to TFT circuits.

Examples of embodiment of such an X-ray imaging device will be described in further detail hereafter in relation with the drawings.

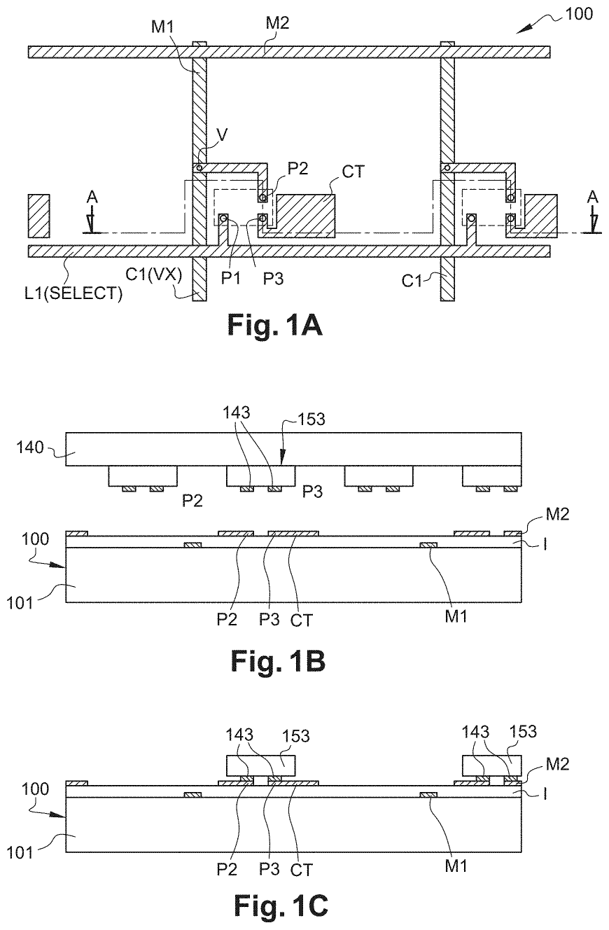

FIGS. 1A, 1B, 1C, 1D, 1E, and 1F are top and cross-section views illustrating steps of an example of a method of manufacturing an X-ray imaging device according to an embodiment.

FIG. 1A is a partial simplified top view of an example of embodiment of the transfer substrate 100 of the imaging device.

In FIG. 1A, only a portion of transfer substrate 100, corresponding to two adjacent pixels of a same row of the imaging device, has been shown.

FIGS. 1B to 1F are cross-section views of the device at different manufacturing stages.

Transfer substrate 100 for example comprises a support plate or sheet 101 made of an insulating material, for example, of glass or of plastic. As a variant, support plate or sheet 101 comprises a conductive support, for example, metallic, covered with a layer of an insulating material. The transfer substrate further comprises electric connection elements, and in particular conductive tracks and conductive pads, formed on the upper surface of support plate 101. These electric connection elements are for example formed by full plate deposition and etching of a succession of conductive and insulating levels on the upper surface of support plate 101. As a variant, the electric connection elements are formed by printing (or another local deposition method) of a succession of conductive and insulating levels on the upper surface of support plate 101.

In the shown example, transfer substrate 100 comprises two conductive metal levels M1 and M2 separated by an insulating level I (not shown in FIG. 1A), and metal vias V (not visible in FIG. 1B) connecting the two metal levels M1 and M2 through insulating level I. In this example, transfer substrate 100 further comprises metal connection pads formed on upper metal level M2, intended to be connected to corresponding connection pads of the elementary chips of the pixels of the device.

Active control circuits of the display device, adapted to powering and controlling the elementary chips of the device via the electric connection elements of the transfer substrate, are for example connected to the electric connection elements of the transfer substrate at the periphery of transfer substrate 100.

As an example, the manufacturing of transfer substrate 100 comprises the three following successive deposition and etching steps.

During a first step, a conductive layer, for example metallic, for example made of titanium, of copper, or of aluminum, is deposited on the upper surface of substrate 101 and then etched to form level Ml. In this example, level M1 comprises a plurality of conductive tracks substantially parallel to the column direction of the array of pixels of the imaging device (vertical direction in the orientation of FIG. 1A). More particularly, in this example, there is formed, in level M1, for each column of the imaging device, a conductive track C1 extending along substantially the entire length of the columns of the device. Each track C1 is intended to convey a signal VX representative of the quantity of electric charges photogenerated in the detectors of the pixels of the corresponding column, and thus of the quantity of X photons received by the detectors of the pixels of the corresponding column.

During a second step, level M1 is covered with a layer of an insulating material, for example, silicon oxide or silicon nitride, to form insulating level I. Local openings are then etched in insulating layer I at the locations of vias V, to enable to establish electric connections between level M1 and level M2. The openings in insulating layer I are for example formed by wet etching, for example, of BHF (“Buffered Hydrofluoric Acid”) type, or by plasma etching.

During a third step, a conductive layer, for example, metallic, is deposited on the upper surface of insulating level I and then etched to form level M2. As an example, the metal layer of level M2 is made of titanium, of copper, or of aluminum. In this example, level M2 comprises a plurality of conductive tracks substantially parallel to the row direction of the array of pixels of the imaging device (horizontal direction in the orientation of FIG. 1A). More particularly, in this example, there is formed in level M2, for each row of the imaging device, a conductive track L1 extending along substantially the entire length of the rows of the device. Each track L1 is intended to convey a signal SELECT for selecting the detectors of the pixels of the corresponding row.

In this example, there is further formed in level M2, for each pixel of the device, a metal region CT, defining a contacting region intended to be electrically connected to a lower electrode of the X-ray detector of the pixel.

After the third deposition step, there are formed, for each pixel, on conductive areas of metal level M2, three metal pads P1, P2, P3, intended to respectively receive three distinct connection pads of the elementary chip of the pixel. Pads P1, P2, P3 are respectively connected to the conductive track L1 of the corresponding pixel row, to the conductive track C1 of the corresponding pixel column, and to contacting region CT.

As a variant, connection pads P1, P2, P3 may be directly formed by portions of level M2.

In this example, pad P1 is connected to the column conductive track C1 of the pixel via a conductive track portion of level M2, pad P2 is connected to row conductive track L1 of the pixel via a conductive track portion of level M2 and a via V, and pad P3 is connected to region CT by a conductive track portion of level M2.

As a non-limiting example, the pitch between pixels of the device in the row direction and in the column direction is in the range from 50 to 500 μm, for example from 100 to 200 μm, for example in the order of 150 μm.

FIGS. 1B and 1C illustrate a step of transfer, in each pixel, of an elementary control and readout chip 153 bonded and electrically connected to the metal connection pads P1, P2, P3 of the pixel.

In this example, elementary chips 153 are collectively transferred from a temporary support substrate 140 to transfer substrate 100.

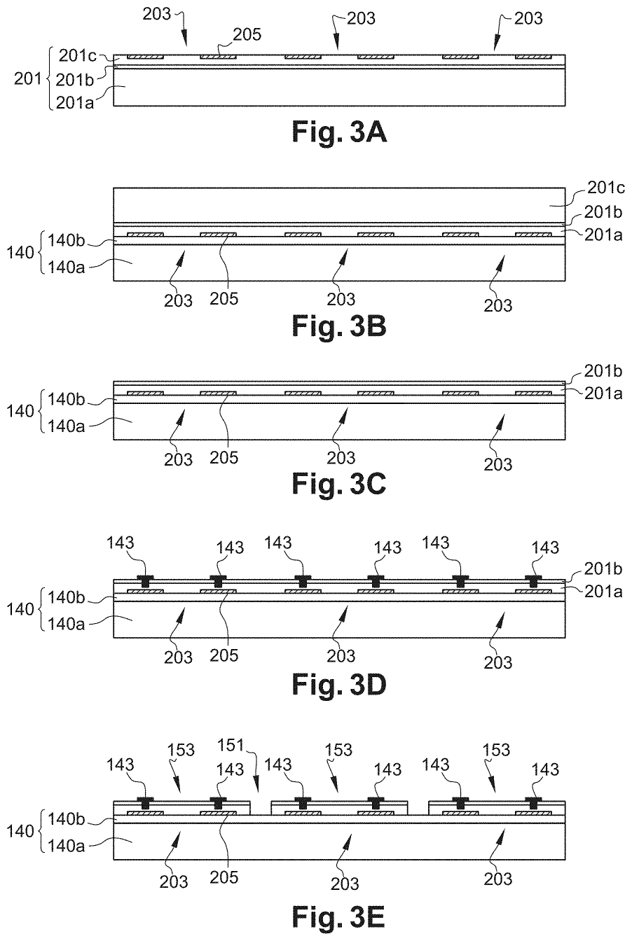

Elementary chips 153 are initially bonded to a surface of temporary support substrate 140 (lower surface in the orientation of the drawings). The structure comprising temporary support substrate 140 and elementary chips 153 is for example formed by a method of the type described hereafter in relation with FIGS. 3A to 3E.

Each elementary chip comprises at least one and preferably a plurality of MOS transistors formed inside and on top of a semiconductor substrate, for example a single-crystal silicon substrate. Elementary chips 153 are for example formed in CMOS technology. Each elementary chip is adapted to delivering, on the column conductive track C1 of the corresponding pixel (via terminal P2), a signal, for example, a voltage, representative of a quantity of X photons received by the X-ray detector of the pixel. Chips 153 may be selected row by row, via the signal SELECT applied to the corresponding conductive track L1, to read the pixels row by row during an image acquisition phase.

Elementary chips 153 are collectively transferred in front of the connection surface of transfer substrate 100, that is, its upper surface in the orientation of the drawings, using temporary support substrate 140 as a handle (FIG. 1B).

Connection pads 143 of elementary chips 153, located on the lower surface side of said chips, are then placed into contact with the corresponding connection pads P1, P2, P3 of transfer substrate 100, and bonded to said connection pads P1, P2, P3. The bonding of the connection pads 143 of elementary chips 153 to the connection pads of the transfer substrate is for example performed by direct bonding, by thermocompression, by soldering, by means of metal microstructures (for example, micropillars) previously formed on pads 143, or by any other adapted bonding and connection method.

Once bonded, by their connection pads 143, to transfer substrate 100, elementary chips 153 are separated from temporary support substrate 140, and the latter is removed (FIG. 1C).

The pitch of the elementary chips 153 on transfer substrate 100 may be greater than the pitch of the elementary chips 153 on temporary transfer substrate 140. Preferably, the pitch of the elementary chips 153 on transfer substrate 100 is a multiple of the pitch of the elementary chips 153 on temporary support substrate 140. In this case, only part of chips 153 is sampled from support substrate 140 at each transfer, as illustrated in FIGS. 1B and 1C. The other chips 153 remain fastened to temporary support substrate 140 and may be used during another step of collective transfer to populate another portion of transfer substrate 100 or another transfer substrate.

FIG. 1D illustrates a step of deposition of a planarization layer 170 on the structure obtained at the end of the steps of FIGS. 1A to 1C. The material of layer 170 is a dielectric material, transparent or not, for example, a polymer material. The material of layer 170 extends from the upper surface of the support substrate, up to a height greater than that of the upper surface of elementary chips 153. Thus, the material of layer 170 entirely covers the support substrate and elementary chips 153. The upper surface of layer 170 is substantially planar and extends continuously over the entire surface of the pixel array.

FIG. 1E further illustrates a step of forming, in each pixel, of a conductive via 301 extending vertically through layer 170. Via 301 is in contact, by its lower surface, with the upper surface of contact region CT. The upper surface of via 301 is flush with the upper surface of layer 170.

FIG. 1F illustrates the structure obtained at the end of a step of transfer of an array detection structure 310 onto the upper surface of passivation layer 170.

Structure 310 comprises an array of X-ray detectors XD of same pitch and of same dimensions as the pixel array of the imaging device. Thus, each pixel of the imaging device comprises a detector XD, located, in this example, vertically in line with the elementary control chip 153 of the pixel.

Each detector XD is formed by a stack of a lower electrode E1, of an active X photon detection stack 103, and of an upper electrode E2.

Electrodes E1 and E2 are for example made of metal or of any other adapted electrically-conductive material to allow the application of an electric field between the electrodes.

Active detection stack 103 comprises at least one layer made of a semiconductor material adapted to directly converting X photons absorbed by the semiconductor material into electric charges (electron/hole pair).

The semiconductor material of detection stack 103 is for example amorphous selenium (a:Se), gallium arsenide (GaAs), mercury iodide (HgI2), lead oxide (PbO), cadmium-zinc telluride (Cd(Zn)Te), or also a perovskite material.

In the shown example, active detection stack 103 and upper electrode supérieure E2 are non-pixelated, that is, they extend continuously over the entire surface of the pixel array of the imaging device.

The lower electrodes E1 of the different detectors are however electrically insulated from one another, to allow an individual reading from the photodetectors XD of the different pixels of the device. In other words, the pixelization of structure 310 is performed at the level of the lower electrodes E1 of detectors XD.

In each pixel of the device, the lower electrode E1 of the pixel detector XD is in contact, by its lower surface, with the upper surface of via 301. Thus, lower electrode E1 is electrically connected to the contacting region CT of the pixel (and thus the elementary chip 153 of the pixel) by means of via 301.

Structure 310 may be formed separately and then transferred onto the upper surface of passivation layer 170. As a variant, electrodes E1, active detection stack 103, and common upper electrode E2 may be successively deposited on the upper surface of passivation layer 170.

Preferably, the lateral dimensions of lower electrodes E1 are greater than 50%, preferably greater than 70%, and more preferably still greater than 90% of the pitch between pixels of the imaging device.

It should be noted that the drawings are not shown to scale. As an example, the active detection stack has a thickness in the range from 200 μm to 1 mm according to the targeted applications, for example in the order of 600 μm. The thickness of elementary chips 153 is for example in the range from 100 to 500 μm. The lateral dimensions of elementary chips 153 are for example in the range from 5 to 150 μm, for example from 10 to 60 μm.

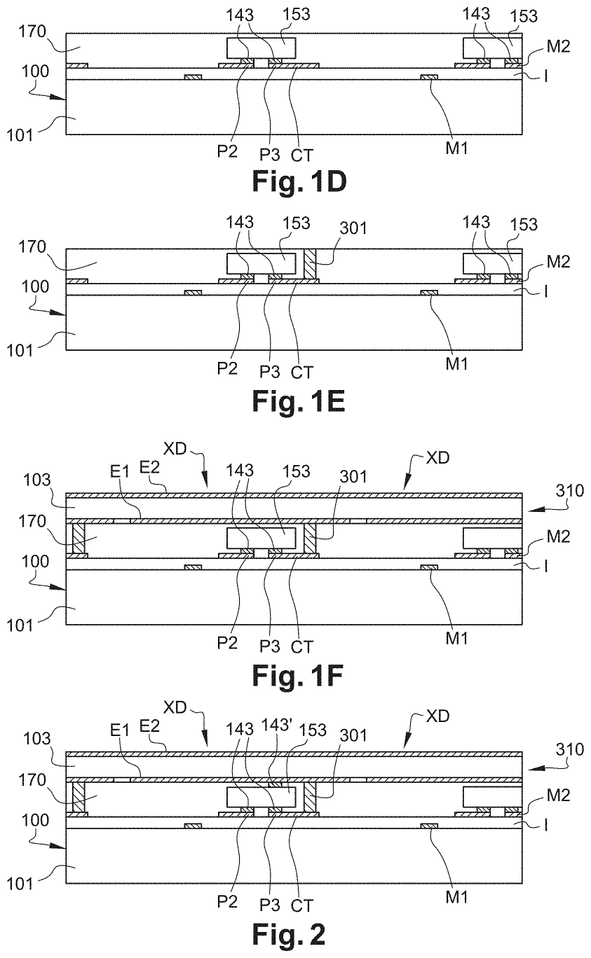

FIG. 2 is a cross-section view illustrating a variant of the method of FIGS. 1A, 1B, 1C, 1D, 1E, and 1F. In this variant, in each pixel, the electric connection between elementary chip 153 and the lower electrode E1 of the detector XD of the pixel is performed via a metal connection terminal 143′ of the elementary chip, located at the upper surface of the elementary chip. Connection terminal 143′ is flush with the upper surface of planarization layer 170. Connection terminal 143′ is in contact, by its upper surface, with the lower surface of electrode E1. Thus, the conductive via 301 and the contact metal region CT of the example of FIGS. 3A to 3F may be omitted.

FIGS. 3A, 3B, 3C, 3D, and 3E are cross-section views illustrating successive steps of an example of a method of manufacturing the elementary chips 153 of an X-ray imaging device of the type described in relation with FIGS. 1A to 1F or 2.

FIG. 3A schematically shows a control structure comprising a first substrate 201 inside and on top of which have been formed a plurality of elementary integrated control circuits 203, for example identical or similar, respectively corresponding to the integrated control circuits of the future elementary chips 153 of the pixels of the device.

In the shown example, substrate 201 is a substrate of SOI (“Semiconductor On Insulator”) type, comprising a semiconductor support substrate 201a, for example, made of silicon, an insulating layer 201b, for example, made of silicon oxide, arranged on top of and in contact with the upper surface of support substrate 201a, and an upper semiconductor layer 201c, for example, made of single-crystal silicon, arranged on top of and in contact with the upper surface of insulating layer 201b.

In this example, elementary control circuits 203 are formed inside and on top of the upper semiconductor layer 201c of substrate 201. Each elementary control circuit 203 for example comprises one or a plurality of MOS transistors (not detailed in the drawings). Elementary control circuits 203 are for example formed in CMOS (“Complementary Metal Oxide Semiconductor”) technology. Each elementary control circuit 203 may comprise a circuit for reading from a photon detector XD of the imaging device.

FIG. 3B illustrates the structure obtained at the end of a step of transfer and of bonding of the structure of FIG. 3A onto temporary support substrate 140.

In FIG. 3B, the orientation of the structure of FIG. 3A is inverted with respect to FIG. 3A.

In this example, temporary support substrate 140 comprises a first layer 140a of a support material, for example, glass or silicon, having a thickness in the range, for example, from 200 to 700 μm, and a second thinner layer 140b made of an adhesive material of relatively low adherence to allow the selective separation of the elementary chips during the step of collective transfer of FIGS. 1B and 1C, for example a polymer material. In this example, layer 140b is arranged on top of and in contact with the upper surface of layer 140a. The structure comprising control circuits 203 is bonded to the upper surface of layer 140b by its lower surface, that is, its surface opposite to support 201a (corresponding to its upper surface in the orientation of FIG. 3A).

FIG. 3C illustrates the structure obtained after a step of removal of the support 101c of the initial SOI structure, for example by grinding and/or chemical etching, to clear the access to the upper surface of the insulating layer 201b of the SOI structure.

It should be noted that the described embodiments are not limited to the above-described example where substrate 201 is an SOI-type substrate. As a variant, substrate 201 may be a solid semiconductor substrate, for example, made of silicon. In this case, at the step of FIG. 3C, substrate 201 may be thinned from its back side (upper surface in the orientation of FIG. 3C), for example, by grinding. An insulating passivation layer, for example made of silicon oxide, may then be deposited on the upper surface of the thinned substrate, replacing layer 201b of the SOI substrate.

FIG. 3D illustrates the structure obtained at the end of steps of forming of contacting openings in layers 201b and 201c, and of forming of contacting metallizations 143 inside and on top of said openings. Metallizations 143 enable to take electric contacts on metal levels (not detailed in the drawings) of the interconnection stack located on the side of the lower surface of semiconductor layer 201c. Metallizations 143 are for example electrically connected to transistors of the control circuit, these transistors being themselves electrically connected or coupled to connection metallizations 205 of circuits 203.

Metallizations 143 form connection terminals of the future elementary chips of the pixels of the device, intended to be connected to corresponding connection terminals of the transfer substrate 100 of the device.

FIG. 3E illustrates the structure obtained at the end of a step of singulation of the elementary pixel chips of the device. For this purpose, trenches 151 extending vertically through layers 201b and 201c are formed from the upper surface of the structure, along sawing lines. In this example, the trenches emerge onto the upper surface of temporary support substrate 140. In top view, trenches 151 form a continuous gate laterally delimiting a plurality of elementary pixel chips 153, for example, identical or similar, each comprising an elementary control circuit 203. Trenches 151 are for example formed by plasma etching.

Elementary chips 153 are intended to be transferred onto the transfer substrate 100 of the X-ray imaging device, as has been described hereabove in relation with FIGS. 1B and 1C.

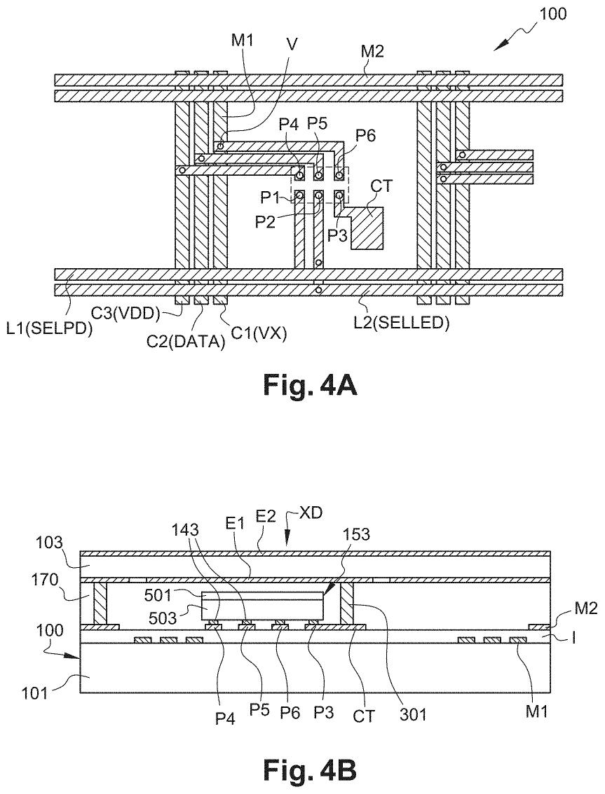

FIGS. 4A and 4B are top and cross-section views illustrating steps of another example of a method of manufacturing an X-ray imaging device according to an embodiment.

FIG. 4A is a partial simplified top view of an example of embodiment of the transfer substrate 100 of the imaging device. FIG. 4B is a cross-section view of the device.

The method of FIGS. 4A and 4B comprises steps identical or similar to the steps of the method of FIGS. 1A to 1F. These steps will not be detailed again hereafter and only the differences with respect to the method of FIGS. 1A to 1F will be highlighted.

The example of FIGS. 4A and 4B differs from the example of FIGS. 1A to 1F mainly in that, in the example of FIGS. 4A and 4B, in each pixel, each elementary chip 153 placed on the transfer substrate comprises not only an integrated circuit for reading from pixel detector XD, but also an inorganic light-emitting diode (LED), and an integrated circuit for controlling the LED.

The integration of a LED in elementary chip 153 advantageously enables to implement, between two image acquisition phases, a step of resetting of detectors XD by application of a light flash on detectors XD.

In this variant, the lower electrodes El of the pixels are preferably made of a transparent conductive material, for example, a transparent conductive oxide, for example indium-tin oxide (ITO).

Elementary chips 153 are monolithic pixel chips, for example, of the type described in the previously-filed patent applications WO2017089676, EP3401958, and WO2018185433. Each chip comprises a LED 501 and an elementary control circuit 503 placed against and electrically connected to the LED. Control circuit 503 is for example made in CMOS technology. Circuit 503 for example comprises a circuit for controlling and reading the X-ray detector XD of the pixel, and a circuit for controlling the LED.

In this example, LED 501 covers the upper surface of elementary control circuit 503. Circuit 503 comprises connection terminal 143 on its lower surface side.

In the example of FIGS. 4A and 4B, planarization layer 170 (FIG. 4B) is made of a transparent material.

To enable to read the electric signals generated by the detectors XD of the pixels and to control the LEDs 501 of the pixels in emission, the transfer substrate 100 of the example of

FIGS. 4A and 4B comprises a number of conductive tracks and of connection metallizations greater than that of the example of FIGS. 1A to 1F. Further, elementary chips 153 comprise a number of connection terminals greater than what has been previously described.

In the shown example, each elementary chip comprises six connection terminals intended to be respectively connected to six metal connection pads P1, P2, P3, P4, P5, P6 of transfer substrate 100.

As an example, level M1 comprises, for each pixel column of the imaging device, three column conductive tracks C1, C2, C3 intended to respectively convey a signal VX representative of the intensity of the X radiation received by the detectors XD of the pixels of the corresponding column, a signal DATA for controlling the LEDs of the pixels of the corresponding column, a signal DATA for controlling the LEDs of the pixels of the corresponding column, and a signal VDD for powering the LEDs of the pixels of the corresponding column. As an example, level M2 comprises, for each pixel row of the imaging device, two row conductive tracks L1 and L2 intended to respectively convey a signal SELX for selecting the X-ray detectors of the pixels of the corresponding row, and a signal SELLED for selecting the LEDs of the pixels of the corresponding column.

Metal pads P1, P2, P3, P4, P5, P6 are formed on conductive areas of metal level M2 and are intended to respectively receive six distinct connection pads of the elementary pixel chip. Pads P1, P2, P3, P4, P5, P6 are respectively connected to the conductive track L1 of the corresponding pixel row, to the conductive track L2 of the corresponding pixel row, to the electrode El of the detector XD of the corresponding pixel, to the conductive track C3 of the corresponding pixel column, to the conductive track C4 of the corresponding pixel column, and to the conductive track C5 of the corresponding pixel column.

It should be noted that the variant of FIGS. 4A and 4B may be combined with the variant of FIG. 2, in which case the connection elements CT and 301 of FIG. 4B may be omitted.

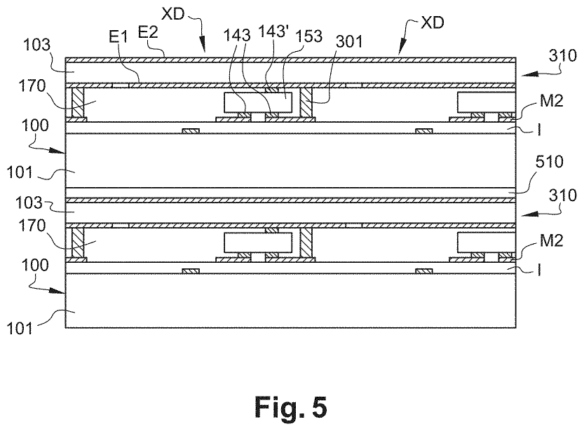

FIG. 5 is a cross-section view illustrating another alternative embodiment of an X-ray imaging device according to an embodiment. In this example, the device comprises two stacked devices of the type described in relation with FIG. 1F.

The device of FIG. 5 enables to perform dual-energy X-ray imaging, also called color X-ray imaging, that is, to respectively image a first energy level, called low-energy level (BE), by means of the upper imaging device, and a second energy level, called high-energy level (HE), by means of the lower imaging device. As an example, the upper imaging device is adapted to detecting radiations having an energy level in the range from 1 keV to 140 keV, for example, from 40 keV to 80 keV, for example in the order of 60 keV in average, and the lower imaging device is adapted to detecting radiations having a wavelength in the range from 60 keV to 140 keV, for example from 80 keV to 120 keV, for example in the order of 100 keV in average.

In the shown example, an interface layer 510 is arranged between the lower surface of the support substrate 101 of the upper device and the upper surface of the detection structure 310 of the lower device. The thickness of the support substrate of the upper device is preferably relatively small to limit the absorption of high-energy photons. As an example, the thickness of the support substrate of the upper device is smaller than the thickness of the support substrate of the lower device.

Interface layer 510 may comprise a filtering layer adapted to filtering the low-energy radiation so that only the high-energy radiation reaches the lower imaging device. The filtering layer is for example a metal layer, for example, continuous, for example, made of copper or of aluminum, for example having a thickness in the range from 0.1 to 0.4 mm. The filtering layer enables to improve the spectral separation between the two imaging devices. Preferably, the filtering layer is electrically insulated from the upper electrode of detection structure 310, for example, by means of a transparent film made of an electrically-insulating material (not detailed in the drawing), for example, a polymer material. As an example, each of the two devices comprises a protection package, not visible in the drawings, for example made of an electrically-insulating material, the filtering layer being arranged between the two devices.

As a variant, interface layer 510 may be omitted.

The embodiment of FIG. 5 may of course be combined with all the previously-described variants.

Various embodiments and variants have been described. Those skilled in the art will understand that certain features of these various embodiments and variants may be combined, and other variants will occur to those skilled in the art. In particular, the described embodiments are not limited to the examples of dimensions and materials mentioned in the present disclosure.

What is claimed is:

1. X-ray imaging device, comprising:
a transfer substrate comprising electric connection elements;
an array of pixels, each comprising a monolithic elementary chip bonded and electrically connected to elements of electric connection of the transfer substrate, and a direct conversion X photon detector electrically connected to the elementary chip, wherein, in each pixel, the elementary chip comprises an integrated circuit for reading from the detector of the pixel.

2. Device according to claim 1, wherein, in each elementary chip, the integrated circuit for reading from the detector of the pixel is formed in CMOS technology.

3. Device according to claim 1, wherein, in each pixel, the detector comprises an active detection stack based on a semiconductor material adapted to directly converting X photons into electric charges, for example, a material from the group comprising amorphous selenium, gallium arsenide, mercury iodide, lead oxide, cadmium-zinc tellurideTe), or a perovskite material.

4. Device according to claim 3, wherein the active detection stack continuously extends over the entire surface of the pixel array.

5. Device according to claim 3, wherein, in each pixel, the detection comprises a lower electrode and an upper electrode, the upper electrode being common to all the pixels of the device and the lower electrode being individualized per pixel.

6. Device according to claim 1, wherein, in each pixel, the detector covers the elementary chip of the pixel.

7. Device according to claim 1, wherein, in each pixel, the elementary chip of the pixel comprises an inorganic LED and an integrated circuit for controlling the LED.

8. Assembly comprising first and second stacked X-ray imaging devices according to claim 1.

9. Assembly according to claim 8, comprising a filtering layer between the first and second devices.

10. Method of manufacturing an X-ray imaging device according to claim 1, wherein the elementary chips are collectively transferred and bonded to the transfer substrate, by means of a temporary support substrate.

11. Method according to claim 8, comprising a step of deposition of a planarization layer after the transfer and the bonding of the elementary chips onto the transfer substrate.

12. Method according to claim 9, comprising a step of transfer of the detectors onto the upper surface of the planarization layer.
